(19) United States
(12) Patent Application Publication
(10) Pub. No.: US 2023/0161923 A1
(43) Pub. Date: May 25, 2023

(54) METHOD AND INTERNET OF THINGS SYSTEM FOR DEPLOYING NUCLEIC ACID DETECTION POINTS IN A SMART CITY

(71) Applicant: CHENGDU QINCHUAN IOT TECHNOLOGY CO., LTD., Chengdu (CN)

(72) Inventors: Zehua SHAO, Chengdu (CN); Yuefei WU, Chengdu (CN); Haitang XIANG, Chengdu (CN); Yaqiang QUAN, Chengdu (CN); Xiaojun WEI, Chengdu (CN)

(73) Assignee: CHENGDU QINCHUAN IOT TECHNOLOGY CO., LTD., Chengdu (CN)

(21) Appl. No.: 18/152,742

(22) Filed: Jan. 10, 2023

(30) Foreign Application Priority Data
Nov. 10, 2022 (CN) 202211401992.X

(51) Int. Cl. G06F 30/13; G06F 30/27; G16B 45/00; G16B 50/00

(52) U.S. Cl. CPC G06F 30/13; G06F 30/27; G16B 45/00; G16B 50/00

(57) ABSTRACT

Embodiments of the present disclosure provide a method and Internet of Things system for deploying nucleic acid detection points in a smart city. This method is executed based on a management platform of the Internet of Things system for deploying nucleic acid detection points in a smart city, comprising: predicting nucleic acid detection person-time in a preset future period in at least one area in multiple areas based on epidemic information and environmental information in multiple areas; determining a deployment plan of the nucleic acid detection points based on the predicted nucleic acid detection person-time.

### CROSS-REFERENCE TO RELATED DISCLOSURES

This application claims priority to Chinese Patent Application No. 202211401992.X, filed on Nov. 10, 2022, the entire contents of which are hereby incorporated by reference.

### TECHNICAL FIELD

The present disclosure relates to the field of information management technology, and in particular to a method and an Internet of Things system for deploying nucleic acid detection points in a smart city.

### BACKGROUND

At present, the prevention and control of the coronavirus epidemic are still very tight, routine nucleic acid testing, as a key step in timely and accurate prevention and control of the epidemic, has been rolled out in many cities. In this context, scientific and reasonable deployment of nucleic acid detection points is very important.

As a huge network formed by various information sensor devices and the Internet, the smart Internet of Things can realize the interconnection of people, machines, and objects at any time and any place, and can provide a technical basis for the deployment of nucleic acid detection points. It is of great significance to design a method and an Internet of Things system for deploying nucleic acid detection points in a smart city.

Therefore, it is hoped that there would be a method and an Internet of Things system for deploying nucleic acid detection points in a smart city, which can effectively deploy limited detection resources, and provide citizens with more convenient nucleic acid detection services as well as implement prevention and control measures.

### SUMMARY

One of the embodiments of the present disclosure provides a method for deploying nucleic acid detection points in a smart city, which is executed based on a management platform of an Internet of Things system for deploying nucleic acid detection points in a smart city, comprising: predicting nucleic acid detection person-time in a preset future period in at least one of multiple areas based on epidemic information and environmental information in the multiple areas; and determining a deployment plan for the nucleic acid detection points based on the predicted nucleic acid detection person-time.

One of the embodiments of the present disclosure provides an Internet of Things system for deploying nucleic acid detection points in a smart city, including an object platform, a sensor network platform, and a management platform; the management platform is configured to perform the following operations: predicting nucleic acid detection person-time in a preset future period in at least one of multiple areas based on epidemic information and environmental information in the multiple areas; the object platform is used to obtain epidemic information and environmental information, and transmit them to the management platform through the sensor network platform; determining a deployment plan for the nucleic acid detection points based on the predicted nucleic acid detection prediction person-time.

### BRIEF DESCRIPTION OF THE DRAWINGS

The present disclosure is further illustrated in terms of exemplary embodiments. These exemplary embodiments are described in detail with reference to the drawings. These embodiments are not limited. In these embodiments, the same number represents the same structure, wherein:

FIG. 1 is a schematic diagram of an Internet of Things for deploying nucleic acid detection points in a smart city according to some embodiments of the present disclosure;

FIG. 2 is a flowchart of the process for deploying nucleic acid detection points in a smart city according to some embodiments of the present disclosure;

FIG. 3 is a schematic diagram of the area map and prediction model structure according to some embodiments of the present disclosure;

FIG. 4 is a flowchart of determining a deployment plan for the nucleic acid detection points according to some embodiments of the present disclosure;

FIG. 5 is a flowchart of determining the position of a mobile detection point according to some embodiments of the present disclosure.

### DETAILED DESCRIPTION

In order to illustrate the technical solutions of the embodiments of the present specification more clearly, the following briefly introduces the accompanying drawings that are used in the description of the embodiments. Obviously, the accompanying drawings in the following description are only some examples or embodiments of the present specification, for those of ordinary skill in the art, the present specification can also be applied to other similar situations according to these drawings without any creative effort. Unless obviously obtained from the context or the context illustrates otherwise, the same numeral in the drawings refers to the same structure or operation.

It will be understood that the terms “system,” “device,” “unit,” and/or “module” used herein are one method to distinguish different components, elements, parts, sections, or assemblies of different levels. However, if other words may achieve the same purpose, the words may be replaced by other expressions.

As shown in the present disclosure and the claims, unless the context clearly suggests exceptional circumstances, the words “one”, “a”, “an” and/or “the” do not specifically refer to the singular form, but may also include the plural form. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

The present disclosure uses the operation of the flow chart for the operation of the system implemented by the implementation example of the present disclosure. It should be understood that the foregoing or following operations may not necessarily performed exactly in order. Conversely, the operations may be implemented in an inverted order, or simultaneously. At the same time, other operations can also be added to these processes, or from these processes to remove a certain step or several steps.

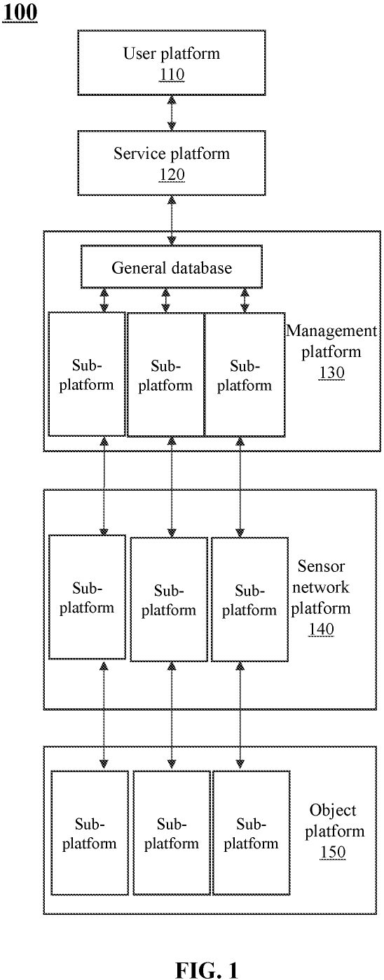

FIG. 1 is a schematic diagram of an Internet of Things for deploying nucleic acid detection points in a smart city according to some embodiments of the present disclosure.

As shown in FIG. 1, the Internet of Things system 100 for deploying nucleic acid detection points in a smart city may include user platform 110, service platform 120, management platform 130, sensor network platform 140, and object platform 150.

User platform 110 refers to a platform led by users, including the platforms that obtain the needs of users and feedback the information to the users. The user platform 110, in some embodiments, is configured to query the deployment plan information of the nucleic acid detection points by inputting an instruction through a user terminal device. In some embodiments, the user platform 110 is configured to display the deployment plan information of the nucleic acid detection point through the user terminal device.

In some embodiments, the user platform 110 may interact downward with the service platform 120. For example, issuing a query instruction for the deployment plan of nucleic acid detection points to the service platform 120; receiving the deployment plan of nucleic acid detection points uploaded by the service platform 120.

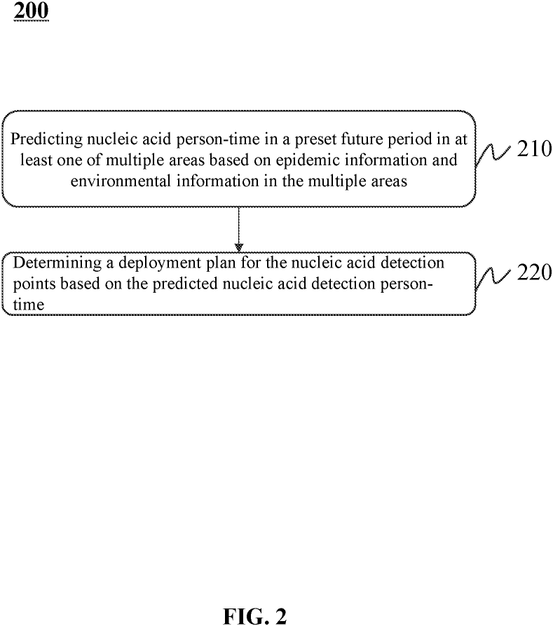

In some embodiments, the deployment plan may include whether to add a fixed/mobile nucleic acid detection point and the specific position if added, see the relevant parts of FIG. 2 for details.

Service platform 120 refers to a platform for the preliminary processing of users’ inquiry needs. In some embodiments, the service platform 120 is configured as the first server and adopts a centralized layout. The centralized layout means that the reception, processing, and transmission of data or/and information are carried out by the platform in a unified manner.

In some embodiments, the service platform 120 may interact downward with the management platform 130. For example, issuing a query instruction for the deployment plan of nucleic acid detection points to the service platform 130, and receiving the deployment plan of nucleic acid detection points uploaded by the management platform 130.

In some embodiments, service platform 120 may interact upward with the user platform 110. For example, receiving a query instruction for the deployment plan of nucleic acid detection points issued by the user platform, and uploading the deployment plan of nucleic acid detection points to user platform 110.

In some embodiments, management platform 130 includes a general management platform database and several management sub-platforms.

In some embodiments, the management platform 130 is a platform for executing the method for deploying nucleic acid detection points in a smart city. In some embodiments, the management platform 130 may be used to respond to the user’s inquiry needs, process the relevant monitoring data of the urban areas uploaded by the sensor network platform, and determine the deployment plan of urban nucleic acid detection points.

In some embodiments, the sub-platforms of management platform 130 process and manage the corresponding data, upload the processed data to the general platform database, and then upload the summarized data to the service platform 120 by the general platform database.

In some embodiments, the sub-platform of the management platform 130 may be a nucleic acid detection point management sub-platform of the urban areas, which may be divided according to urban areas and corresponding to the sensor network sub-platform.

In some embodiments, the data interaction of management platform 130 includes: each management sub-platform may receive monitoring data from the corresponding sensor network sub-platform; each management sub-platform may process and operate the monitoring data in each area. For example, the monitoring-related data of the A area is uploaded to the A area’s nucleic acid detection points management sub-platform for management, and the monitoring-related data of the B area is uploaded to the B area’s nucleic acid detection points management sub-platform for management, each management sub-platform may further upload the processed data to general database of the management platform; the general database of the management platform may upload the monitoring data to the service platform 120 after summary processing, and the data uploaded to the service platform 120 may include nucleic acid detection points deployment plan in each area of the city.

In some embodiments, the management platform 130 may interact downward with the sensor network platform 140. For example, receiving the monitoring data of each area uploaded by the sensor network platform 140 to process, and issuing instructions for obtaining monitoring data to the sensor network platform 140.

In some embodiments, management platform 130 may interact upward with service platform 120. For example, receiving nucleic acid detection points deployment plan query instructions issued by service platform 120, and uploading the nucleic acid detection points deployment plan to service platform 120.

In some embodiments, by processing monitoring data of different areas through management sub-platforms, and summarizing to the general database, the processing pressure of the entire management platform 130 can be reduced, and the data of the independent management sub-platforms can be aggregated for unified management.

In some embodiments, the sensor network platform 140 refers to the platform that passes monitoring data of areas to management platform 130. In some embodiments, sensor network platform 140 is configured as a communication network and gateway. Each platform is configured with independent gateways and adopts an independent layout. In some embodiments, the sensor network platform 140 includes several sensor network sub-platforms, which are divided according to urban areas and correspond to several sub-platforms of the management platform.

In some embodiments, the data interaction of sensor network platform 140 includes: the monitoring-related data may be processed and managed in the corresponding sub-platform. For example, the relevant monitoring equipment in A area uploads the monitoring data to the sensor network sub-platform in A area, and the relevant monitoring equipment in B area uploads the monitoring data to the sensor network sub-platform in B area; the sensor network sub-platform uploads the processed monitoring data to the corresponding management sub-platform.

In some embodiments, sensor network platform 140 may interact downward with object platform 150. For example, receiving the monitoring-related data uploaded by object platform 150, and issuing monitoring-related data instructions to object platform 150.

In some embodiments, sensor network platform 140 may interact upward with management platform 130. For example, receiving an instruction for obtaining monitoring-related data issued by the management sub-platform, and uploading the monitoring-related data of the sensor network sub-platform to the corresponding management sub-platform, etc.

In some embodiments, object platform 150 may be a functional platform for sensor information generation and controlling the final execution of information. In some embodiments, object platform 150 includes several object sub-platforms, and each object sub-platform obtains monitoring data applied to one area; several object sub-platforms are separately corresponding to several sensor network sub-platforms.

In some embodiments, object platform 150 may be configured as various monitoring equipment. For example, the monitoring equipment may be a camera, which may obtain images in areas (such as determining pedestrian flow situation and crowd gathering situation). In some embodiments, the camera has a unique logo and may be used to deploy in different urban areas. In some embodiments, monitoring equipment may be personnel terminal devices, and may be used to obtain personnel’s positioning information and environmental information. Wherein, environmental information can include the population and population distribution, etc. See the relevant parts of FIG. 2 for details.

In some embodiments, object platform 150 may interact upward with sensor network platform 140. For example, receiving the instructions for obtaining monitoring data issued by the sensor network sub-platform, and uploading monitoring data to the corresponding sensor network sub-platform.

It should be noted that the above description of the system and its components are only for the convenience of description, and cannot limit the scope of the implementation of the present disclosure to the scope of the embodiments. It may be understood that for those skilled in the art, after understanding the principle of the system, it is possible to arbitrarily combine the various parts, or form a subsystem to connect with other parts without departing from the principle. For example, management platform 130 and service platform 120 may be integrated into one component. For another example, each part may share one storage device, and each part may also have its own storage device. Such deformations may be all within the scope of the protection of the present disclosure.

FIG. 2 is a flowchart of the process for deploying nucleic acid detection points in a smart city according to some embodiments of the present disclosure. As shown in FIG. 2, process 200 includes the following steps. In some embodiments, process 200 may be performed by management platform 130.

Step 210, predicting nucleic acid detection person-time in a preset future period in at least one of multiple areas based on epidemic information and environmental information in the multiple areas.

Area may refer to a certain area. For example, a city, a community, etc. In some embodiments, the management platform may determine multiple areas in various ways. In some embodiments, the area may correspond to the administrative division area. For example, the multiple areas may include Chengdu, Deyang, and Nanchong. For another example, the multiple areas may include Gaoxin District, Qingyang District, Jinjiang District, etc. For another example, the multiple areas may include Beson Community, Baohe Community, and Xingfu Community.

In some embodiments, multiple areas may also be divided based on the roads of the city. For example, the areas on both sides of a road are respectively divided into area 1 and area 2.

It can be understood that the management platform may also determine a larger or smaller area based on different needs and/or tasks, which may be determined according to the actual application scenario.

Epidemic information refers to information related to the epidemic of Coronavirus. For example, information related to the outbreak of the epidemic, information related to epidemic infected personnel, etc. In some embodiments, the epidemic information may include areas of historical epidemic outbreaks and the scale of outbreaks.

Areas of historical epidemic outbreaks refer to areas where Coronavirus cases have been diagnosed during the historical time before the current time. For example, Chengdu Gaoxin District has been diagnosed with cases of Coronavirus before the current time, which makes it become an area of historical epidemic outbreaks.

The scale of outbreaks refers to the severity reflected in the epidemic, wherein the severity is related to the case scope of the epidemic and the number of diagnoses. The larger the case scope of the epidemic, the larger the number of cases, the greater the severity, and the larger the outbreak.

Environmental information may include the population, population distribution, and the population in and out of the area within a preset time period. In some embodiments, environmental information may include information related to the population conditions and facilities in a certain range. For example, the environmental information of the Chengdu Gaoxin District may include information related to the population conditions and facilities in the Chengdu Gaoxin District.

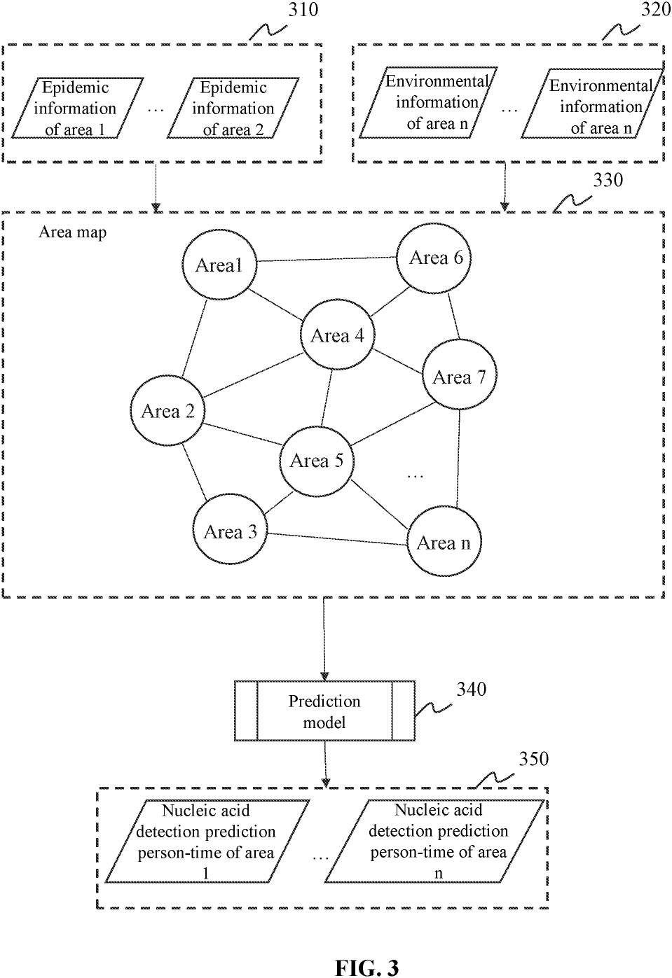

In some embodiments, the pedestrian flow situation in an area may be obtained based on environmental information. Wherein, the pedestrian flow situation may include pedestrian volume, foreign population, local permanent population, and population activity. For more descriptions of the pedestrian flow situation, please refer to FIG. 3 and its description.

The population refers to the total population in a certain area within a certain time period. For example, the population of Chengdu in 2022 was 2.1192 million. In some embodiments, the population may include local permanent population, migrant permanent population, and the population of foreign visitors. Wherein, the local permanent population may refer to the number of people whose household registration is in the local area and who often live in the local area, and the migrant permanent resident population may refer to the number of people whose household registration is in other places and who often live in the local area. For example, the local permanent population in Chengdu may refer to the number of people whose household registration is in Chengdu and who often live in Chengdu, and the migrant permanent resident population in Chengdu may refer to the number of people whose household registration is outside Chengdu and who often live in Chengdu. For more descriptions of foreign visitors to the population, please refer to FIG. 3 and its description.

The population distribution (also referred to as distribution of population)refers to the distribution status of the population in a certain geographical space within a certain time period. For example, in 2022, the population distribution of Chengdu was 16.844 million urban population and 4.356 million rural population. In some embodiments, the population distribution may include the distribution of population amount and the distribution of population quality. Wherein, the distribution of population quality may include the distribution of population in gender, age, education level, occupation, etc. For example, in 2022, the population age distribution of the Chengdu Gaoxin District may be under 1000,000 under 30 years old, 1.3 million from 30 years old to 60 years old, and 700,000 over 60 years old.

In some embodiments, the management platform may obtain epidemic information and environmental information through big data analysis. For example, the management platform may obtain a large amount of data through telecommunications operator data and network crawling for statistical analysis.

In some embodiments, the management platform may obtain epidemic information and environmental information through third-party platform analysis. In some embodiments, the management platform may obtain epidemic information through national and/or local epidemic information release platforms. For example, the management platform may obtain the area of historical epidemic outbreaks and the scale of outbreaks of the historical epidemic in Chengdu through the Chengdu epidemic information release platform. In some embodiments, the management platform may obtain environmental information through national and/or local government service platforms. For example, the management platform may obtain the population and population distribution of Chengdu through the Chengdu government service platform.

In some embodiments, the management platform may predict nucleic acid detection person-time in a preset period in at least one of multiple areas based on epidemic information and environmental information in multiple areas.

The preset future time period may refer to the time interval after the current time point set according to the actual situation, for example, 48 hours after the current time, or 14 days after the current time.

The nucleic acid detection person-time may refer to the sum of the number of people who would take nucleic acid detection within the preset future period. For example, if the number of people to be tested for nucleic acid in the next 48 hours is 1 million and the frequency is 2 times, the predicted nucleic acid detection person-time (also referred to as nucleic acid detection prediction person-time hereinafter) in the next 48 hours is 2 million.

In some embodiments, the management platform may organize historical data information such as historical epidemic information, historical environment information, and historical nucleic acid detection prediction person-time in multiple areas as a data comparison table, and determine nucleic acid detection prediction person-time based on the data control table. For example, based on the data comparison table, the historical epidemic situation in Chengdu Gaoxin District is outbreak scale of 3 cases, the historical environment is 2.5 million population, and the historical nucleic acid detection prediction person-time in the historical period is 5 million within 48 hours. When the epidemic information is outbreak scale of 3 cases and the environmental information is 2.8 million population, the nucleic acid detection prediction person-time in the next 48 hours may be 5.6 million.

In some embodiments, the management platform may also determine the nucleic acid detection prediction person-time through a prediction model.

In some embodiments, based on the epidemic information and environmental information of multiple areas, predicting the nucleic acid detection prediction person-time in at least one of the multiple areas in a preset future time period may include processing the epidemic information and environmental information of the multiple areas by a prediction model, and predicting the predicted nucleic acid detection prediction person-time in at least one area.

In some embodiments, the prediction model may be a machine learning model. In some embodiments, the prediction model may include the Deep Neural Network (DNN) model, the Convolutional Neural Network (CNN) model, the recurrent neural network (RNN) model, etc, or combinations of such models.

In some embodiments, the input of the prediction model may be the epidemic information and environmental information in multiple areas, and the output of the prediction model may be nucleic acid detection prediction person-time in at least one area.

In some embodiments, the prediction model may be obtained through training. For example, input training samples to the initial prediction model, establish a loss function based on the output results of the label and initial prediction model, and update the parameters of the initial prediction model. When the loss function of the initial prediction model satisfies a preset condition, the training of the model is completed, wherein the preset condition may be that the loss function converges and the number of iterations reaches a threshold.

In some embodiments, the training sample may be the historical epidemic information and historical environment information of multiple areas, and the training sample may be obtained based on historical data. The label of the training sample may be the nucleic acid detection person-time in multiple areas. The label may be manually marked. For more descriptions of the prediction of nucleic acid detection by prediction models, please refer to FIG. 3 and its description.

The methods described in some embodiments of the present disclosure may accurately predict the nucleic acid detection prediction person-times in at least one area in a preset future time period through a prediction model based on epidemic information and environmental information of multiple areas. It can be more accurate to predict the nucleic acid detection prediction person-time in conjunction with the actual situation, reduce the human cost and resources required for artificial predictions, and make the prediction process of nucleic acid detection predictions more efficient.

Step 220, determining a deployment plan for the nucleic acid detection points based on the predicted nucleic acid detection person-time.

The deployment plan may refer to the planning and arrangement scheme of settings nucleic acid detection points. For example, the deployment plan of nucleic acid detection points may include whether to add a fixed/mobile nucleic acid detection point and the specific position of the nucleic acid detection point when adding it.

In some embodiments, the management platform may determine the deployment plan of nucleic acid detection points based on nucleic acid detection prediction person-time. For example, if the maximum service person-time of the existing nucleic acid detection points in Chengdu Gaoxin District in the next 48 hours is 2.3 million, and the nucleic acid detection prediction person-time in Chengdu Gaoxin District in the next 48 hours is 2.8 million, the deployment plan of nucleic acid detection points may add 500,000 service person-time to Chengdu Gaoxin District, and the service areas of each nucleic acid detection point may cover the whole Chengdu Gaoxin District after merging. Wherein the maximum service person-time refers to the maximum number of times that nucleic acid detection points within a certain range can provide nucleic acid detection services within a preset time period.

In some embodiments, the maximum service person-time may be determined based on the resource allocation of nucleic acid detection points. Service area refers to the geographical distribution area of the population served by nucleic acid detection points. For example, the service area of nucleic acid detection points of a street is the administrative area of the street. In some embodiments, the service area may be determined based on the historical data of each nucleic acid detection point. For example, based on the geographical distribution area of the population served by a certain nucleic acid detection point in a certain historical time, the distribution area is determined as the service area of the nucleic acid detection point.

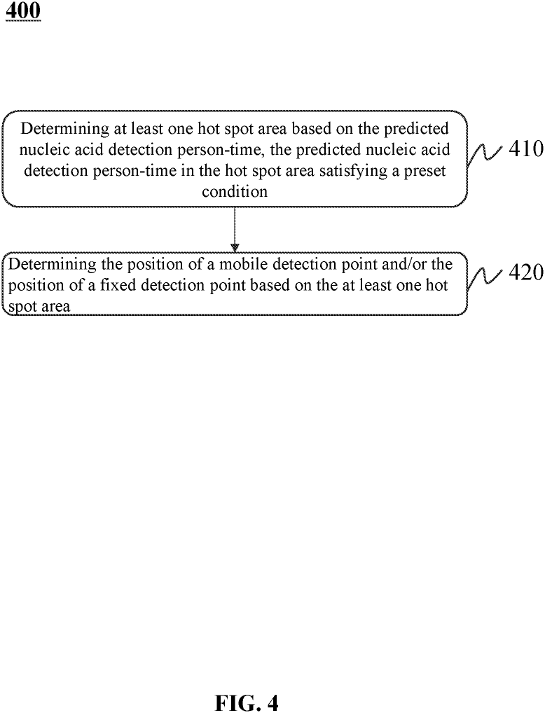

In some embodiments, determining the deployment plan of nucleic acid detection points based on the nucleic acid detection prediction person-time may include determining at least one hot spot area based on the nucleic acid detection prediction person-time, the nucleic acid detection prediction person-time in the hot spot area meeting a preset condition; determining the position of a mobile detection point and/or the position of a fixed detection point based on the at least one hot spot area. In some embodiments, the position of a mobile detection point is between at least two hot spot areas. For more descriptions of the position of a mobile detection point and/or the position of a fixed detection point, please refer to FIG. 4 and its instructions.

Some embodiments of the present disclosure obtain the epidemic information and environmental information of multiple areas through the management platform, and predict nucleic acid detection prediction person-time in a preset future period in at least one of multiple areas, which can obtain more accurate and more effective nucleic acid detection prediction person-time, and based on the nucleic acid detection prediction person-time, determining the deployment plan of the nucleic acid detection points, which can improve the scientificity and rationality of the deployment of nucleic acid detection points, and provide more convenient nucleic acid detection services for regional personnel, which is conducive to timely discovery and control the source of infection and implement prevention and control measures faster.

FIG. 3 is a schematic diagram of the area map and prediction model structure according to some embodiments of the present disclosure.

In some embodiments, prediction model 340 is a diagram neural network model.

In some embodiments, predicting nucleic acid detection prediction person-time in a preset future period in at least one of multiple areas based on epidemic information 310 and environmental information 320 in multiple areas including building an area map 330 based on the epidemic information 310 and environmental information 320 of the multiple areas, and predicting the nucleic acid detection prediction person-time 350 in the area corresponding to the nodes based on the processing of the area map 330 by the prediction model 340.

In some embodiments, the input of the neural network model is the area map 330, and the output is the nucleic acid detection prediction person-time 350 of areas corresponding to prediction nodes. The output of the neural network model may be the nucleic acid detection prediction person-time corresponding to each node, and the nucleic acid detection prediction person-time of the nodes in this area may be determined according to the corresponding nodes in the vector. The output of the neural network model may be a vector composed of the nucleic acid detection prediction person-time in each area. For example, the vector of the output of the neural network model is (1000,1500,1200,1300), which indicates the nucleic acid detection prediction person-time corresponding to area node 1, area node 2, area node 3, area node 4 is 1,000, 1500, 1200, and 1300. The nucleic acid detection prediction person-time in the area may be the nucleic acid detection prediction person-time nucleic acid detection points in the area.

In some embodiments, the graph neural network model may be obtained through training. For example, input training sample to the initial graph neural network model, and establish a loss function based on the output results of a label and initial graph neural network model, update the parameters of the initial graph neural network model. When the loss function of the initial graph neural network model satisfies a preset condition, the model training is completed, wherein the preset conditions may be that the loss function converges, the number of iterations reaches a threshold.

In some embodiments, the training sample may be several historical area maps, and the training sample may be obtained based on historical data. The label of the training sample may be the nucleic acid detection person-time in the area. Labels may be manually marked.

Area map 330 may refer to a semantic network diagram constructed based on epidemic information and environmental information based on multiple areas. As shown in FIG. 3, area map 330 may include node data and edge data.

Node data may include area nodes and corresponding node features. The area node of the area map corresponds to the area where the crowd gathering situation in multiple areas satisfies a preset requirement, and each node corresponds to one area. As shown in FIG. 3, an area map includes area 1, area 2, ..., area N. For details of areas, please refer to the details of the present disclosure, such as FIG. 2.

The crowd gathering situation may refer to the pedestrian volume in an area. The pedestrian volume may refer to the density of the population in an area. The degree of density may be represented by the proportion of the population and the acreage of an area. For example, the smaller the area and the more population, the higher the pedestrian volume in the area. The population of the area may include the sum of the local permanent population and the population of foreign visitors. Foreign visitors may refer to the non-local permanent population visitors in an area.

Crowd gathering situation satisfies a preset requirement may refer to that the pedestrian volume in this area exceeds a preset crowd gathering threshold. For example, the crowd gathering threshold is 0.1 people/m², if the pedestrian volume of area A and area B is 0.2/m² and 0.01 people/m², then area A may be used as an area node.

In some embodiments, node features may include whether there is a nucleic acid detection point, allocation of nucleic acid detection resources, a pedestrian flow situation within a preset distance range, and the epidemic information of the node in the area.

The allocation of nucleic acid detection resources refers to the allocation of resources required for nucleic acid detection. It may be represented by the number of people who can be detected in nucleic acid detection points in the area within the preset time period. For example, the allocation of nucleic acid detection resources in a certain area A can provide 1000 times nucleic acid detection services for area A within 1 day. Correspondingly, the allocation of nucleic acid detection resources in area A is 1000. When there is no nucleic acid detection point in the area, the allocation of the corresponding nucleic acid detection resources can be 0.

In some embodiments, whether the area is provided with nucleic acid detection points and the allocation of nucleic acid detection resources based on the arrangement of existing nucleic acid detection points in the environmental information may be determined; the epidemic information of the area may be determined based on the epidemic information.

The pedestrian flow situation may refer to the situation related to the population flow during a preset time period. In some embodiments, the pedestrian flow situation may include at least one of the pedestrian volume, foreign population, local permanent population, and population activity.

The local permanent population may include the population that lives within the area or within a preset distance range around the area. For example, in scenic spots A, the local permanent population may refer to the population of the preset range near scenic area A. The local permanent population may be the population of the area, including the local population in the area and the resident foreign population.

The foreign population may refer to the number of people who enter the area to visit. For example, the population of foreign population may refer to the count of tourists to scenic area A, and the foreign population may also refer to the count of people of visiting friends in the residential area B. In some embodiments, the foreign population may include local permanent residents who have traveled to and returned to the city where the area is located within the city during a preset time period. For example, Personnel A is a local permanent population of Beijing residential area A. If personnel A arrives in Langfang City today and returns to residential area A, that makes personnel A a foreign population. In some embodiments, the foreign population may also include local permanent residents who have traveled to other key areas (e.g., high-risk areas of epidemic) and returned within a preset time period. For example, doctor A is the local permanent population of Beijing residential area A. Doctor A needs to reach residential area B for nucleic acid detection, and residential area B is a high-risk area of epidemic, which makes doctor A a foreign population. In some embodiments, the foreign population may be a population visiting persons from other areas.

Population activity may refer to the activity of the population in and out of the area within the preset time period. In some embodiments, since there may be a step of scanning health codes before entering a certain area, the population activity may be expressed by the number of people scanning health codes in this area in a preset time. In some embodiments, the population activity in this area is directly proportional to the number of people scanning health codes in this area in the preset time period. The more people who scan health codes in a preset time period, the higher the population activity in this area.

Based on node features, the nucleic acid detection person-time in this area may be estimated. The higher the pedestrian flow, the population, the local permanent population, and population activity, the higher the nucleic acid detection prediction person-time.

In some embodiments, the pedestrian flow situation in the area may be determined based on the population quantity, population distribution, and population activity in environmental information.

In some embodiments, the pedestrian volume may be obtained based on mobile terminals. The population of this area may be determined by the positioning of the mobile terminals and the boundary of this area, and the ratio of the population of this area to the area of this area may be further determined to get the pedestrian volume in this area.

The edge data of the area map includes edges and corresponding features. In some embodiments, the edge of the area map corresponds to the road between areas, that is, if there is a direct way between two areas, there is an edge between the nodes corresponding to the two areas to connect the two areas. The features of the edge include the distance between the areas. For example, area nodes A and area node B correspond to community 1 and community 2, and there is a road (Jianlin Road) between community 1 and community 2, then the feature of the edge connecting area node A and area node B is the distance between Jianlin Road in community 1 and community 2. In some embodiments, if there are several roads between two areas, then the features of the edge connecting the nodes corresponding to the two areas include the length of the road with the shortest distance.

In some embodiments, the edge data of the area map can be determined by the road information in the area in the environmental information.

By determining the areas with and without nucleic acid detection points as the nodes of the area map, and setting whether there are nucleic acid detection points and the allocation of nucleic acid detection resources in the node features, it is convenient to add nucleic acid detection points according to the analysis results, and the positions of newly added nucleic acid detection points can be quickly determined to satisfy the requirements. By setting the pedestrian flow situation within the preset distance nearby in the node features of the area map, it is easy to know the pedestrian flow situation in and out of the region. By setting the distance between the areas of the area map, the positions of the mobile detection points that are added later can be determined, and the positions of the mobile detection points can be in line with the actual situation. Based on the map structure, analysis of multiple areas at the same time, the efficiency of operation can be improved, making the processing of determining nucleic acid detection prediction person-time more efficient.

FIG. 4 is a flowchart of determining a deployment plan for the nucleic acid detection points according to some embodiments of the present disclosure. In some embodiments, process 400 may be executed by the management platform.

Step 410, determining at least one hot spot area based on the predicted nucleic acid detection person-time, the predicted nucleic acid detection person-time in the hot spot area satisfying a preset condition.

The preset condition that the nucleic acid detection prediction person-time satisfies may be that the nucleic acid detection prediction person-time exceeds the corresponding set threshold. Hot spot area may refer to the area where the nucleic acid detection prediction person-time exceeds the threshold number of times. For example, if the person-time threshold is 1,000, and the nucleic acid detection prediction person-time of the area A is 1100, then area A is a hot spot area.

Step 420, determining the position of a mobile detection point and/or the position of a fixed detection point based on the at least one hot spot area.

The mobile detection point may refer to the nucleic acid detection point where the position can be moved. For example, the mobile detection point may be a nucleic acid detection vehicle.

The fixed detection point may refer to the nucleic acid detection point fixed. For example, a nucleic acid detection window opened by a community hospital.

The position of the detection point may be set in an area, or between two adjacent areas.

In some embodiments, different thresholds may be set for the differences between the nucleic acid detection prediction person-time at the detection points in an area and the preset nucleic acid detection person-time thresholds at the corresponding detection points, respectively, such as setting a first threshold and a second threshold, and whether to set a fixed detection point or a mobile detection point may be further determined, wherein the second threshold is higher than the first threshold, and the preset number of times of detection at each detection point can be set manually. The preset nucleic acid detection person-time threshold of each detection point may be related to the allocation of nucleic acid detection resources in that detection point, for example, it may be equal to or slightly smaller than the detectable number of people corresponding to the allocation of nucleic acid detection resources.

In some embodiments, based on the differences between the nucleic acid detection prediction person-time at the detection points in an area and the preset nucleic acid detection person-time thresholds at the corresponding detection points, respectively the detection point deployment plan in the area may be determined. When the difference between the nucleic acid detection prediction person-time and the preset nucleic acid detection person-time threshold of the detection point exceeds the first threshold, and if there is no detection point in the area, a fixed detection point is required to be added in the area.

When the difference between the nucleic acid detection prediction person-time at the detection point in this area and the preset nucleic acid detection person-time threshold of the detection point exceeds the second threshold, it is further necessary to set up mobile detection points around this area to satisfy the nucleic acid detection requirements of this area.

In some embodiments, the settings of a fixed detection point and mobile detection point may be based on the principle of convenience. Wherein, the set position of the mobile detection point may also be further combined with the difference between the nucleic acid detection prediction person-time in the detection point of adjacent areas and the preset nucleic acid detection person-time threshold of the detection point. For example, as shown in FIG. 3, the area with roads between area 4 includes the area 1, 2, 5, 6, and 7, wherein if the difference between the nucleic acid detection prediction person-time at the detection point in area 7 and the preset nucleic acid detection person-time threshold of the detection point is the largest, the mobile detection point may be set on the road between area 4 and area 7.

In some embodiments, the position of the mobile detection point is between at least two hot spot areas. In some embodiments, two hot spot areas may be two adjacent hot spot areas. After determining a hot spot area, another hot spot area adjacent to it may be determined based on the area map, and then the position of the mobile point between the two adjacent hot spot areas may be set. For example, for 4 hot spot areas, the position of the mobile detection point may be set at the intersection of the connecting lines of the non-adjacent hot spot areas in the 4 hot spot areas.

In some embodiments, when the two hot spot areas are two non-adjacent hot spot areas, the position of the mobile detection point between the two hot spot areas may be set in the area between the two hot spot areas. For example, there is an area node C between hot spot area A and hot spot area B, and the position of the mobile detection point may be located in C. In some embodiments, the mobile detection point between two hot spot areas may be set in the middle position between the two hot spot areas.

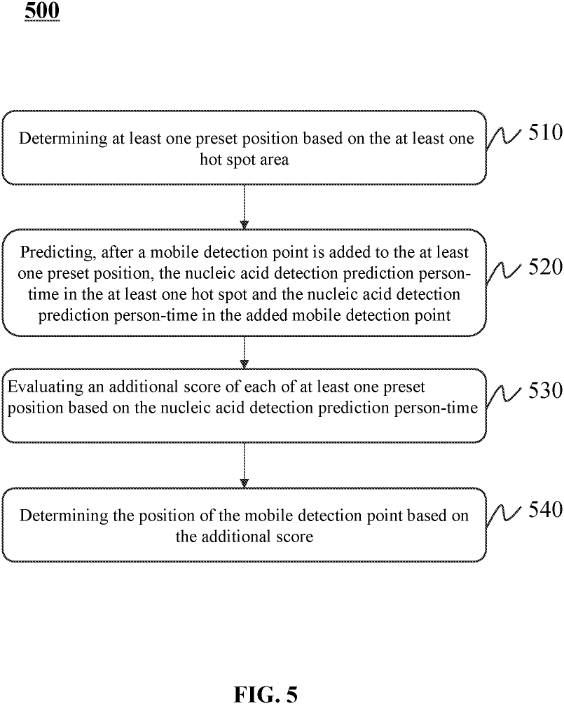

In some embodiments, based on at least one hot spot area, determining the position of a mobile detection point includes: determining at least one preset position based on at least one hot spot area; predicting, after a mobile detection point being added to the at least one preset position, the nucleic acid detection prediction person-time in the at least one hot spot area and the nucleic acid detection prediction person-time in the added mobile detection point; evaluating an additional score of at least one preset position in every preset position based on the nucleic acid detection prediction person-time; determining the position of the mobile detection point based on the additional score. For more details on determining the position of the mobile detection point, please refer to the description of other parts of the present disclosure, such as FIG. 5.

By adding a mobile detection point, the pressure of nucleic acid detection in hot spot areas can be relieved, thus satisfying the needs of users for nucleic acid detection and improving the efficiency of nucleic acid detection.

FIG. 5 is a flowchart of determining the position of a mobile detection point according to some embodiments of the present disclosure. In some embodiments, process 500 can be performed by the management platform.

Step 510, determining at least one preset position based on the at least one hot spot area.

The preset position may be a position where the mobile detection point may be added.

In some embodiments, the preset position may be a position between the two hot spot areas or around the hot spot area. The preset position may be multiple. For example, if there are two areas around the hot spot area, that is area A and area B, the preset position may include the location on the road to area A and the location on the road to area B.

In some embodiments, the preset position may correspond to an area, which may include an area where the crowd gathering situation does not satisfy the preset conditions or an area on the area map where the nucleic acid detection prediction person-time does not satisfy the preset conditions. For example, there is an area B around the hot spot area A that is not a node on the area map, and the preset position can be the area B.

In some embodiments, the preset position may be located at different positions on the same road between two hot spot areas, or it may also be located at different positions on different roads between two hot spot areas. For example, there are roads C and D between hot spot areas A and B, the preset position can be located on road C or on road D.

Step 520, predicting, after a mobile detection point is added to the at least one preset position, the nucleic acid detection prediction person-time in the at least one hot spot area and the nucleic acid detection prediction person-time in the added mobile detection point.

In some embodiments, the nucleic acid detection prediction person-time in at least one hot spot area and the nucleic acid detection prediction person-time in the newly added mobile detection point may be predicted by the updated area map.

The updated area map may refer to the area map composed of a new area node corresponding to the preset position in the original area map. For example, the original area map includes area node A, area node B, and area node C, and the area corresponding to the preset position is area D, as a result, the updated area map includes area node A, area node B, area node C, and area node D. The node features in the updated area map are similar to the node features in the original area map. The detailed content of the node features and edge features in the updated area map can be referred to the description of other parts of the present disclosure, for example, description of the area map in FIG. 3.

In some embodiments, the prediction model may be used to process the updated area map, predict the nucleic acid detection prediction person-time in the areas corresponding to the nodes in the updated area map, where the prediction model may be a graph neural network model. For details of the prediction model, please refer to the other parts of the present disclosure, such as FIG. 3.

In some embodiments, the input of the neural network model is the updated area map, and the output is the nucleic acid detection prediction person-time corresponding to the nodes in the updated area map. In some embodiments, the nucleic acid detection prediction person-time corresponding to the nodes in the updated area map may include updated nucleic acid detection prediction person-time in the original area and the nucleic acid detection prediction person-time in the additional detection points. For example, the original hot spot area node is area node A. When area node B is newly added, it is necessary to re-determine the nucleic acid detection prediction person-time of area node A and area node B. After adding nucleic acid detection points, the nucleic acid detection prediction person-time in the original hot spot areas may be reduced.

Through the updated area map, it can grasp the influence of adding nucleic acid detection points around the original hot spot area on the nucleic acid detection prediction person-time of the detection points in the original hot spot area.

Step 530, evaluating an additional score of each of at least one preset position based on the nucleic acid detection prediction person-time.

Additional score may refer to the score of the position of the additional detection point. The addition score can be used to indicate the degree of mitigation of nucleic acid detection in the original hot spot area by adding detection points. The additional score may be a score or other forms.

In some embodiments, the additional score of the preset position is related to the auxiliary prediction data.

Auxiliary prediction data may refer to data used to determine the additional score. The auxiliary prediction data includes the nucleic acid detection prediction person-time in the preset position, and the nucleic acid detection prediction person-time of at least two of the hot spot areas whose distances from the preset position satisfy a preset distance condition. The preset distance condition may not exceed the preset distance threshold, for example, the preset distance threshold may not exceed 5 km.

The nucleic acid detection prediction person-time in each area may be determined by a prediction model. The distance between the position and the preset position of the hot spot area may be obtained through the area map.

In some embodiments, the additional score may include additional score determined based on the distance and the additional score determined based on the nucleic acid detection prediction person-time.

In some embodiments, when there are multiple preset positions, an additional score determined based on the distance may be obtained based on the sum of the distances (absolute values) between each preset position and the positions of two hot spot areas. When the sum of the distances is higher, the lower the corresponding position of the preset position. For example, there are preset positions C and D between hot spot areas A and B, and the sum of the distances between position C and hot spot areas A and B is 100 meters, and the sum of the distances between position D and hot spot areas A and B is 150 meters, so the additional score of the preset position D is low. The additional score determined based on the distance can be determined by corresponding the difference of the distance to the addition scores in different stages.

In some embodiments, when there are multiple preset positions, an additional score determined based on the nucleic acid detection prediction person-time may be obtained based on the sum of absolute values of the difference between the nucleic acid detection prediction person-time of each detection point (including the detection point in the original hot spot area and the detection point in the preset position) and the preset detection person-time threshold of each detection point. When the sum of the absolute values of the differences is the smallest, the highest the additional score of the corresponding preset position. The sum of the absolute values of the differences can be correlated with the additional scores of different score segments to determine the additional scores determined based on the nucleic acid detection prediction person-time. For example, if the sum of absolute values of the differences is 0-200, the corresponding additional score is 90; if the sum of absolute values of the differences is 200-400, the corresponding additional score is 80.

For example, there are preset positions C and D between hot spot areas A and B, the preset detection person-time threshold of hot spot area A is 1,000, and the preset detection person-time threshold of hot spot area B is 1200. After adding a detection point in preset position C, the nucleic acid detection prediction person-time in hot spot area A will be reduced to 900, and the nucleic acid detection prediction person-time in hot spot area B will be reduced to 1,000, the nucleic acid detection prediction person-time and the preset detection person-time threshold of the mobile detection point at the position C are 500 and 600, respectively, the sum of the absolute values of the difference between the nucleic acid detection prediction person-time at the preset position C and the threshold of the preset nucleic acid detection prediction person-time at the respective detection points is (1000-900)+(1200-1000)+(600-500)=400; after a detection point has been added at the preset position D, the nucleic acid detection prediction person-time in hot spot area A is reduced to 950, and the nucleic acid detection prediction person-time in hot spot area B is reduced to 1050. The nucleic acid detection prediction person-time in preset position D is 600, and the preset detection point threshold of the mobile detection point threshold at the preset position D is 600, the sum of the absolute values of the difference between the nucleic acid detection prediction person-time at the preset position C and the preset nucleic acid detection prediction person-time threshold at the respective detection points is (1000-900)+(1200-1050)+(600-600)=200; then correspondingly, the additional score in the preset position C is higher than the additional score of the preset position D.

In some embodiments, different weights may be set for the additional score determined based on the distance and the additional score determined based on the nucleic acid detection prediction person-time, so as to determine the additional score. For example, the additional score determined based on the distance is 80, the weight is 0.4, and the additional score determined based on the nucleic acid detection prediction person-time is 90 and the weight is 0.6, so the additional score is 86.

Based on additional score, it can be determined whether there is a need to add a detection point in the preset position, and the best position if added.

Step 540, determining the position of the mobile detection point based on the additional score.

In some embodiments, the position of the mobile detection point may be set at the additional position with the highest additional score.

Through the overall assessment of the nucleic acid detection prediction person-time changes after the mobile detection point, it is determined whether to add mobile detection point and how many the mobile detection points are added, which satisfies the actual situation and avoids the waste of public resources. Further, by calculating the reasonable positions of mobile detection points, the efficiency of nucleic acid detection can be improved and the user needs can be satisfied.

It should be noted that different embodiments may have different beneficial effects. In different embodiments, the possible beneficial effects may be any of the above or the like, or any combination thereof, or any other possible beneficial effects.

Having thus described the basic concepts, it may be rather apparent to those skilled in the art after reading this detailed disclosure that the foregoing detailed disclosure is intended to be presented by way of example only and is not limiting. Although there is no clear explanation here, those skilled in the art may make various modifications, improvements, and corrections for the present disclosure. This type of modification, improvement, and corrections are recommended in the present disclosure , so this class is corrected, improved, and the amendment remains in the spirit and scope of the exemplary embodiment of the present disclosure.

Meanwhile, the present specification uses specific words to describe the embodiments of the present specification. For example, “one embodiment,” “an embodiment,” and/or “some embodiments” mean that a certain feature, structure, or characteristic is connected with at least one embodiment of the present disclosure. Therefore, it is emphasized and should be appreciated that two or more references to “an embodiment” or “one embodiment” or “an alternative embodiment” in various parts of the present disclosure are not necessarily all referring to the same embodiment. Further, certain features, structures , or features of one or more embodiments of the present specification can be combined.

Furthermore, unless explicitly stated in the claims, the order of processing elements and sequences, the use of alphanumerics, or the use of other names described in the present disclosure is not intended to limit the order of the processes and methods of the present specification. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, the present disclosure method does not mean that the features needed in the spectrum ratio of this disclosure ratio are more characteristic. Rather, claimed subject matter may lie in less than all features of a single foregoing disclosed embodiment.

In some embodiments, numbers expressing quantities of ingredients, properties, and so forth, configured to describe and claim certain embodiments of the application are to be understood as being modified in some instances by the term “about,” “approximate,” or “substantially”. Unless otherwise stated, “about”, “approximate” or “substantially” indicates that the number of numbers allows ± 20%of changes. Correspondingly, in some embodiments, the value parameters used in the present disclosure and claims are approximate values. The approximate values may be changed according to the features of individual embodiments. In some embodiments, the numerical parameters should consider the effective digits specified and use a general digit reservation method. Although the numerical domains and parameters used in the present disclosure are used to confirm its range breadth, in the specific embodiment, the settings of such values are as accurate as possible within the feasible range.

For each patent, patent application, patent application publications and other materials cited by the present disclosure, such as articles, books, instructions, publications, documents, etc., all of them will be incorporated in the present disclosure as a reference. Except for the application history documentation of the present disclosure or conflict, there is also an except for documents (currently or after the present disclosure ) in the widest range of documents (currently or later). It should be noted that, if there is any inconsistency or conflict between the descriptions, definitions, and/or usage of terms in subsidiary information the present disclosure and the contents of the present disclosure, the descriptions, definitions, and/or usage of terms in the present disclosure shall prevail.

Finally, it should be understood that the embodiments described in the present disclosure are intended to illustrate the principles of the embodiments of the present disclosure. Other deformations are also possible within the scope of the present disclosure. Therefore, as an example rather than restrictions, the replacement configuration of the embodiment of the present disclosure may be consistent with the teaching of the present disclosure. Correspondingly, the embodiments of the present disclosure are not limited to the implementation and description of the present disclosure.

What is claimed is:

1. A method for deploying nucleic acid detection points in a smart city, which is executed based on a management platform of an Internet of Things system for deploying nucleic acid detection points in a smart city, comprising:
predicting nucleic acid detection person-time in a preset future period in at least one of multiple areas based on epidemic information and environmental information in the multiple areas; and
determining a deployment plan for the nucleic acid detection points based on the predicted nucleic acid detection person-time.

2. The method of claim 1, wherein the predicting nucleic acid detection person-time in a preset future period in at least one of multiple areas based on epidemic information and environmental information in the multiple areas comprises:
predicting nucleic acid detection person-time in the at least one area based on the processing of the epidemic information and environmental information of the multiple areas by a prediction model, the prediction model being a machine learning model.

3. The methods of claim 2, wherein the prediction model is a graph neural network model, and the predicting nucleic acid detection person-time in a preset future period in at least one of multiple areas based on epidemic information and environmental information in the multiple areas comprises:
building an area map based on the epidemic information and environmental information of the multiple areas, nodes of the area map corresponding to areas where a crowd gathering situation meets a preset requirement, and edges of the area map corresponding to roads between the areas; and
predicting the nucleic acid detection person-time in the areas corresponding to the nodes based on the processing of the area map by the prediction model.

4. The method of claim 3, wherein a node feature of each node comprises: whether there is a nucleic acid detection point, allocation of nucleic acid detection resources, and a pedestrian flow situation within a preset distance range, wherein the pedestrian flow situation includes at least one of pedestrian volume, foreign population, local permanent population, and population activity; an edge feature of each edge is a distance between the areas corresponding to two connected nodes.

5. The method of claim 4, wherein the pedestrian volume is obtained based on a mobile terminal.

6. The method of claim 1, wherein the determining a deployment plan for the nucleic acid detection points based on the predicted nucleic acid detection person-time comprises:
determining at least one hot spot area based on the predicted nucleic acid detection person-time, the predicted nucleic acid detection person-time in the hot spot area satisfying a preset condition; and
determining a position of a mobile detection point and/or a position of a fixed detection point based on the at least one hot spot area.

7. The method of claim 6, wherein the position of the mobile detection point is located between at least two hot spot areas.

8. The method of claim 7, wherein the determining the position of the mobile detection point based on the at least one hot spot area comprises:
determining at least one preset position based on the at least one hot spot area;
predicting, after the mobile detection point is added to the at least one preset position, nucleic acid detection person-time in the at least one hot spot area and nucleic acid detection person-time in the added mobile detection point;
evaluating an additional score of each of the at least one preset position based on the predicted nucleic acid detection person-time in the at least one hot spot area and in the added mobile detection point; and
determining the position of the mobile detection point based on the additional score.

9. The method of claim 8, wherein the additional score of the preset position is related to auxiliary prediction data, and the auxiliary prediction data includes the predicted nucleic acid detection person-time at the preset position, and predicted nucleic acid detection person-time in at least two of the hot spot areas whose distances from the preset position satisfy a preset distance condition.

10. The method of claim 1, wherein the Internet of Things system for deploying nucleic acid detection points in a smart city further comprises a user platform, a service platform, a sensor network platform, and an object platform;
the object platform is used to obtain epidemic information and environmental information, and transmit them to the management platform through the sensor network platform; and
the method further comprises:
transmitting, based on the service platform, the deployment plan for the nucleic acid detection points to the user platform.

11. The method of claim 1, wherein the management platform includes a general database of the management platform and several management sub-platforms; the sensor network platform includes several sensor network sub-platforms; different sensor network sub-platforms and different management sub-platforms correspondingly transmit and/or process data of different areas.

12. An Internet of Things system for deploying nucleic acid detection points in a smart city comprising an object platform, a sensor network platform, and a management platform; the management platform is configured to perform the following operations including:
predicting nucleic acid detection person-time in a preset future period in at least one of multiple areas based on epidemic information and environmental information in the multiple areas; the epidemic information and the environmental information of the multiple areas being obtained based on the object platform, and transmitted to the management platform based on the sensor network platform;
determining a deployment plan for the nucleic acid detection points based on the predicted nucleic acid detection person-time.

13. The Internet of Things system of claim 12, wherein the management platform is further configured to perform the following operations including:
predicting nucleic acid detection person-times in the at least one area based on the processing of the epidemic information and environmental information of the multiple areas by a prediction model, the prediction model being a machine learning model.

14. The Internet of Things system of claim 13, wherein the prediction model is a graph neural network model, and the management platform is further configured to perform the following operations including:
building an area map based on the epidemic information and environmental information of the multiple areas, nodes of the area map corresponding to areas where a crowd gathering situation meets a preset requirement, and edges of the area map corresponding to roads between the areas; and
predicting the nucleic acid detection person-time in the areas corresponding to the nodes based on the processing of the area map by the prediction model.

15. The Internet of Things system of claim 14, wherein a node feature of each node comprises: whether there is a nucleic acid detection point, allocation of nucleic acid detection resources, and a pedestrian flow situation within a preset distance range, wherein the pedestrian flow situation includes at least one of pedestrian volume, foreign population, local permanent population, and population activity; an edge feature of each edge is a distance between the areas corresponding to two connected nodes.

16. The Internet of Things system of claim 15, wherein the management platform is further configured to perform the following operations including:
determining at least one hot spot area based on the predicted nucleic acid detection person-time, the predicted nucleic acid detection person-time in the hot spot area meeting a preset condition; and
determining a position of a mobile detection point and/or a position of a fixed detection point based on the at least one hot spot area.

17. The Internet of Things system of claim 16, wherein the position of the mobile detection point is located between at least two hot spot areas.

18. The Internet of Things system of claim 17, wherein the management platform is further configured to perform the following operations including:
determining at least one preset position based on the at least one hot spot area;
predicting, after the mobile detection point is added to the at least one preset position, nucleic acid detection person-time in the at least one hot spot area and nucleic acid detection person-time in the added mobile detection point;
evaluating an additional score of each of at least one preset position based on the predicted nucleic acid detection person-time in the at least one hot spot area and in the added mobile detection point; and
determining the position of the mobile detection point based on the additional score.

19. The Internet of Things system of claim 18, wherein the additional score of the preset position is related to auxiliary prediction data, and the auxiliary prediction data includes the predicted nucleic acid detection person-time at the preset position, and predicted nucleic acid detection person-time in at least two of the hot spot areas whose distances from the preset position satisfy a preset distance condition.

20. The Internet of Things system of claim 12, wherein the Internet of Things system for deploying nucleic acid detection points in a smart city further includes a user platform and a service platform;
the service platform is used to transmit the deployment plan for the nucleic acid detection points to the user platform.
